(19) United States
(12) Patent Application Publication
(10) Pub. No.: US 2011/0137342 A1
(43) Pub. Date: Jun. 9, 2011

(54) Button Anchor System for Moving Tissue

(75) Inventors: James Henderson, Gatineau (CA); Michael T. O'Malley, Appleton (CA); Timothy Maxwell, Carp (CA); Bert Reitsma, Manotick (CA)

(73) Assignee: CANICA DESIGN INC.

(21) Appl. No.: 13/030,900

(22) Filed: Feb. 18, 2011

Related U.S. Application Data
(63) Continuation of application No. 11/223,324, filed on Sep. 8, 2005, now abandoned.
Application No. 11/223,324 is a continuation-in-part of application No. PCT/IB2001/000796, filed on Nov. 15, 2001.
Application No. PCT/IB2001/000796 is a continuation-in-part of application No. 10/192,326, filed on Jul. 9, 2002, now Pat. No. 7,361,185.
(60) Provisional application No. 60/608,686, filed on Sep. 9, 2004.

(51) Int. Cl. A61B 17/04

(52) U.S. Cl. 606/232

(57) ABSTRACT

A system of non-reactive components for moving or for moving and stretching plastic tissue that exerts a relatively constant dynamic force over a variety of distances and geometries, that is easily adjustable, and is self-adjusting. This system includes a “button anchor system” for moving tissue, particularly including deep fascia and muscle layers of the abdominal or thoracic cavity wall, in surgical, post surgical, and post traumatic reconstruction where the wound margins are beyond a distance that permits normal re-approximation. Button anchor assemblies allow re-approximation of severely retracted abdominal wall and full thickness thoracic wounds where a closure force is required to be applied to the sub-dermal layers. Systems of this invention allow for such a force to be applied and externally controlled during treatment.

### RELATED APPLICATION DATA

This application is a continuation of U.S. patent application Ser. No. 11/223,324, filed Sep. 8, 2005, which claims priority to U.S. Provisional Patent Application No. 60/608,686, filed Sep. 9, 2004, and is a continuation-in-part of International Application No. PCT/IB01/00796, filed Nov. 15, 2001, published in English under publication No. WO 01/85035; and is a continuation-in-part of U.S. patent application Ser. No. 10/192,326, filed Jul. 9, 2002, published under publication No. 2003/0092969, all of which are herein incorporated by this reference.

### FIELD OF THE INVENTION

This invention relates generally to a system and method for moving or for moving and stretching human or animal plastic tissue that exerts a relatively constant tension over a given distance and that is readily adjustable, and more specifically to an anchor for use with such systems.

### BACKGROUND

In general, surgery and surgical treatment involve one or both of tissue separation and tissue joining. In surgery, medical treatment, and medical research, it is desirable to retract tissue, stabilize tissue, and present tissue in a variety of specific orientations to provide access to the area under investigation or repair, ideally in a method that creates minimal trauma beyond what is necessary for exposure and visualization of the operative area. Ultimately, the procedures should allow for immediate, or primary, closure of the wound. Unfortunately, the latter option is not always available in surgical or trauma wound scenarios.

Moving tissue presents unique challenges, as tissues often resist joining, or closure, depending on the nature of the tissue structure, the circumstances of the tissue separation, and general patient health. Complications related to wound closure and healing generally result from major forces, minor forces and/or compromised healing responses. Major forces are retractive forces created beyond the viscoelastic properties of the tissue, and may be created by: (1) increased internal volume, such as in the case of obesity, which elevates containment forces on the skin system; (2) changes in aspect ratio, such as increased abdominal circumference created in prone, non-ambulatory patients due to muscular atrophy; (3) respiratory muscular activity; (4) muscular response; (5) loss of fascia structure; (6) muscular-skeletal deformation; (7) fleshy appendages; (8) tumors; and (9) severe burns.

Minor forces are internal forces created by the viscoelastic properties of the tissue, which can cause the skin to retract. Elastic tissues, such as skin, comprised mostly of extracellular matrix (ECM) components along with cells, return to a minimum elastic, or relaxed, state when released from tension. In this relaxed state, tissue tensions are minimized and balanced. Skin tissue in this minimum elastic state will remain relaxed, demonstrating behavior similar to a non-elastic material. The force required to elongate the tissue in this state often approaches a force that will rupture or sheer structural connective elements, causing localized failures or tears. Soft tissue in this minimum elastic state provides minimum surface coverage and has the highest reluctance to stretch. It is known that a gentle but constant force below the sheer force threshold applied to tissue in combination with adequate hydration will, over time, restore certain tissues to near-original or original elastic state. Additionally, this force can be applied to stretch tissue past the point of equilibrium (normal elastic range) to the maximum elastic range and create the thinnest possible configuration, covering the maximum surface area. If tensions in the tissue do not exceed the point at which the connective structural elements are compromised, the tissue remains at the maximum elastic state as healthy tissue, and normal biological processes will allow cell regeneration and associated ECM production to restore normal skin thickness and tension, which is described below as biological creep.

Plastic tissues, such as skin and muscle, possess certain viscous and elastic rheological properties, and are therefore viscoelastic. Certain plastic tissues are able to increase surface area over time, which can be termed “creep.” “Mechanical creep” is the elongation of skin with a constant load over time beyond intrinsic extensibility, while “biological creep” refers to the generation of new tissue due to a chronic stretching force. A constant and unrelenting force applied to a body tissue, such as skin or muscle, may result in both mechanical and biological creep. Mechanical creep restores the tension originally present but lost in the skin across the incision or wound by retensioning skin, thereby increasing skin coverage. Biological creep occurs more slowly and involves the creation of new tissue. Tissue expansion has long been part of the art of plastic surgery, traditionally accomplished with balloon-type tissue expanders embedded under the skin and externally inflated and increased over time to create expanded pockets of skin for procedures such as breast reconstruction after radical mastectomies, and stretching healthy tissue prior to plastic surgery for the creation of flaps for soft tissue closure.

Finally, compromised healing responses may complicate wound closure or healing. A surgical or other incision becomes a complicated wound as soon as it falls behind normal healing progression. Wound management, including treatment and care of large skin defects and severely retracted incisions, is an area of increasing importance to the health care community. An aging population and an increase in diseases related to obesity and inactivity have increased the occurrence of complicated wounds and placed an increased burden on health care resources. Factors contributing to compromised wound healing include patient age, weight, nutritional status, dehydration, blood supply to the wound site, immune response, allergies to closure materials, chronic disease, debilitating injuries, localized or systemic infection, diabetes, and the use of immunosuppressive, corticosteroid or antineoplastic drugs, hormones, or radiation therapy. Complicated wounds include, but are not limited to: surgical wounds, diabetic ulcers and other chronic ulcers; venous stastis ulcers; decubitis or pressure sores or ulcers; burns; post traumatic lesions, cutaneous gangrene, crush wounds with ischemic necrosis; wounds having exposed plates or bones; keloids; skin lesions; blunt abdominal trauma with perforations; and other acute, subacute or chronic wounds. Treatment and care of these tissue defects is challenging due to difficulties in closure of open wounds.

Two common methods of closure of wounds and skin defects include split thickness skin grafting and gradual closure. A split thickness skin graft involves removing a partial layer of skin from a donor site, usually an upper leg or thigh, and leaving a portion of the dermis at the donor site to regenerate and re-epithelialize. In this manner, a viable skin repair patch can be transferred or grafted to cover a wound area. The graft is often meshed, (which involves cutting the skin in a series of rows of offset longitudinal interdigitating cuts) allowing the graft to stretch to cover two or three times greater an area as well as provide wound drainage while healing. Normal biological function of the skin heals the holes after the graft has been accepted. A meshed graft of this type requires a smaller donor area than a conventional non-meshed or full thickness skin graft. However, these methods do not provide optimal cosmesis or quality of skin cover. Other disadvantages of this method include pain at the donor site, creation of an additional disfiguring wound, and complications associated with incomplete “take” of the graft. In addition, skin grafting often requires immobilization of the limb, which increases the likelihood of contractures. The additional operation and prolongation of hospital stay is an additional economic burden.

Gradual, or progressive, closure is a second method of closure. This technique may involve suturing vessel loops to the wound edge and drawing them together with large sutures in a fashion similar to lacing a shoe. In addition, the wound edges may be progressively approximated with suture or sterile paper tape. The advantages of this gradual, or progressive, technique are numerous: no donor site is required for harvest of a graft, limb mobility is maintained, and superior cosmetic result, more durable skin coverage, better protection from full skin thickness and the maintenance of normal skin sensation may all be achieved.

Existing devices for effecting a gradual closure have many disadvantages. Current methods and devices draw wound edges together using mechanical devices such as screw-actuated systems that require repeated periodic adjustment because a relatively small skin movement substantially eliminates much of the closure force. Widely used existing closure techniques involve use of relatively inelastic materials, such as sutures or surgical tape. Excessive tension may cut the skin or cause necrosis due to point loading of the tissue. Current solutions include suture bolsters, suture bridges, use of staples as anchors at the wound edge, and the use of ligature wire to distribute the load along the wound margins. These approaches all rely on static ribbon or suture material, which must repeatedly be readjusted in order to function effectively, and even with this constant readjustment, maintenance of near constant tension over time is difficult, if not impossible, to achieve. Widely used traditional gradual closure methods rely on static force through fixed distance reduction, and do not provide continuous or dynamic tension.

Many current methods of open wound reduction employ static or non-yielding devices such as sutures or hard approximators, which reduce the distance between the wound margins and rely on the skin's natural elasticity to compensate for movement. One problem with these devices has been that when they are at the point of being most effective, when the skin is at the point of maximum stretch, additional skin tension created through motion, such as breathing or walking, creates stress points where the mechanical fasteners meet the wound margins, causing tearing and wound edge necrosis. This has generally required patients to remain immobile during the course of treatment. Existing systems for treating animals need not consider cosmetic result to such a degree as the healthy patient typically masks the wound site with fur, but cosmesis is a critical criteria in the measurement of a successful result from the system in the human application.

One existing method for effecting closure of a wound utilizes a constant tension, low-grade force to draw wound edges together. One device for practicing this method includes a pair of hooks carried by a pair of sliders that move along a path pulled by a pair of springs. This spring device is enclosed in a plastic housing and is available having various curvatures. The sharp hooks used in this system may damage the skin. The constant force used is a dictated force that is not variable. Other closure devices use elastomeric material, including rubber bands and other types of compressive and non-compressive materials, to approximate wound margins. One kit requires bonding to the skin with an adhesive and also requires periodic adjustment to tighten the straps. Other known closure devices use hooks and elastic loops, which must be replaced with smaller elastic loops to maintain tension, or a motor power source to provide a tightening means. Finally, another current device consists of two surgical needles, two U-shaped lexan polycarbonate arms with hooks on the bottom surface, a threaded tension bar and a polycarbonate ruler. The needles are threaded along the wound margin and each arm is positioned above a needle, with the hooks piercing the skin and engaging the needles. The tension bar is then locked, and tension can be adjusted using the screw.

Existing methods of gradual wound closure fail to provide an effective gradual closure that restores original skin tensions lost across the wound. For example, one system has a single tension of 460 grams. In many instances, such as with the elderly or with compromised skin, this force is too great, resulting in localized failures, tears and necrosis. Many current devices are cumbersome, restrict patient mobility, must be completely removed for wound dressing and cleaning, and are usable in a relatively limited number of situations because of size constraints. Many also require a surgeon for reinstallation after removal for wound dressing. Finally, many current devices cannot readily be used for radial closure of wounds due to their limited ability to pull in a single direction along an overhead beam, thereby restricting their application to parallel pulls along the same axis.

### SUMMARY OF THE INVENTION

This invention provides manipulation and control of tissue positions and tensions on a living person or animal, utilizing both tissue stretch and creep to restore and move tissues. This invention provides methods and devices for moving or for moving and stretching tissue that are simple, easy to use, cost-effective, extremely versatile, self-adjusting and capable of exerting relatively constant force or tension over a variety of distances and at various intersecting angles in wounds having simple or complex geometry.

Components of this invention exert a dynamic force on the tissue, providing and maintaining a maximum safe counter-traction pressure or force across a wound margin or other area. Systems of this invention create controlled constant and unrelenting tension, which can be applied to counteract major or minor retraction forces or to achieve maximum mechanical and biological yields to move or to move and stretch plastic tissue, including closure of large retracted skin defects.

Terms used herein are generally defined and, in some cases, abbreviated, as they are introduced. For convenience, selected terms are also defined here. A force applying component (“fac”) generally stores energy in a manner that exerts force and transmits the force. An elastic force applying component (“efac”) combines these two functions in a single elastic component. The tissue manipulation system of this invention utilizes facs coupled to force coupling components (“anchors”) that couple to tissue the force exerted by the force applying component. The term “elastomer” refers to relatively elastic material, such as silicone, or latex rubber. The term “non-reactive” is used to describe components that are either immunologically inert or hypoallergenic.

Coupling a fac to tissue can occur simply by passing a fac or a portion of a fac such as a suture through a hole created to penetrate tissue. However, such rudimentary coupling works poorly for several reasons, importantly including the extremely poor force distribution across the tissue and the absence of any practical means for adjusting the force exerted by the suture over a period of time.

Anchors of this invention include structures for coupling to force applying components that permit quick, easy attachment and reattachment of various facs, particularly including facs made of silicone, which is extremely difficult to secure. Anchors of this invention provide distribution of force applied and bolster tissue proximate holes through which an fac passes.

This invention provides advances over current methods for moving or moving and stretching plastic tissue through the introduction of gradual but unrelenting tension that is readily adjustable. When tension adjustment is required, it can be accomplished quickly, and the force applying components can include an easily read quantitative visual indicator. Utilizing dynamic force to move and stretch tissue offers the advantage of a relentless countertraction force, while allowing for expansion and contraction of the wound site, which greatly enhances patient mobility and is compliant with respiratory movements.

This invention can be used to apply dynamic force for closure or remodeling of tissue to close dermal wounds, incisions, or defects that may be associated with a variety of conditions, as well as in the stretching of healthy skin in preparation for a skin graft, flap or other remodeling procedure. In one example, this invention includes a system of button anchor assemblies for moving or for moving and stretching plastic tissue, particularly including deep fascia and muscle layers of the abdominal or thoracic cavity wall, in surgical, post surgical, and post traumatic reconstruction where the wound margins are beyond a distance that permits normal re-approximation.

Prior patent applications assigned to Canica Design Inc. describe in detail the use of elastomers and anchors to move and stretch tissue. While the structures disclosed are highly effective, this invention extends the principles disclosed in the earlier patent applications to additionally provide different anchors for the re-approximation of severely retracted abdominal wall and full thickness thoracic wounds where a closure force is required to be applied to the sub-dermal layers. Systems of this invention allow for such a force to be applied and externally controlled during treatment.

### BRIEF DESCRIPTION OF THE DRAWINGS

FIG. 1 is a perspective view of a system of this invention for moving tissue.

FIG. 2 is a perspective view of a button anchor and anchor tail of the system of FIG. 1.

FIG. 3 is a top view of the button anchor of FIG. 2.

FIG. 4 is a front view of the button anchor of FIG. 2.

FIG. 5 is a top view of the anchoring portion of the button anchor of FIG. 2.

FIG. 6 is a top view of the anchor pad of the button anchor of FIG. 2.

FIG. 7 is a perspective view of the anchor pad of the button anchor of FIG. 2.

FIG. 8 is a top view of the anchor tail of the button anchor of FIG. 2.

FIG. 9 is an enlarged detail perspective view of a portion of the anchoring portion of the button anchor of FIG. 2 showing the anchor tail locking interface.

FIG. 10 is a perspective view of installation of part of the system of FIG. 1.

### DETAILED DESCRIPTION

Anchors of this invention are used to transmit and distribute force to the tissue to be moved or stretched. A force applying component according to this invention may be formed in rods, cords, bands, loops, sheets, nets, wires, strands, cables, tubes or other suitable structure. In one embodiment, the fac is an elastic strand that flattens out at the point of maximum load and becomes load dissipating. In one embodiment, a rod-shaped fac is driven through the tissue using a cannula-like device and is attached at each end to an anchor.

Force applying components (“facs”) of this invention may have elastic properties “efacs”) and may be made from any suitable elastomeric material, including, without limitation, latex rubber, silicone, natural rubber and materials of similar elasticity, GR-S, neoprene, nitrile-butyl-polysulfide, ethylene-polyurethane, polyurethane, or any other suitable material that exhibits the property of exerting a return force when held in an elongated state at tensions and distances that are useful in the context of this invention. Efacs may provide a dynamic opposing force equal to or greater than the naturally occurring elastomeric traction forces of the tissue. The efacs of this invention generally are not endless loops but rather are lengths of a single strand, sometimes called a “monostrand,” and may be either solid or hollow. In some instances, multiple strands or endless loops or bands may be used. Significantly, the efacs used in practicing this invention may be secured to a tissue attachment structure at virtually any point along the efac, providing variable tension within the elastic limits of the elastomer used. Use of a non-reactive fac is generally desirable. Non-reactive facs include components that are either immunologically inert or hypoallergenic, such a elastomers formed from silicone or a hypoallergenic form of latex rubber.

Elastomers having various durometers may be used for the force applying components of this invention. Although other elastomeric materials and sizes of material may be used, polyurethane, thermoplastic (TPE) or rubber elastomer in monofilaments 1 mm-8 mm in diameter have been found to be useful in practicing this invention.

In one embodiment, an efac has a 0.125 inch diameter with a nominal durometer of 40. Other efacs, such as efacs having a smaller diameter, may also be provided and differentiated one from another based on color. Alternative shapes, sizes and strengths may be appropriate in some situations. An extruded silicone efac may have a durometer of 40 (which allows a 5:1 stretch ratio). A molded silicone efac may have a durometer of 5 (which allows a 12:1 stretch ratio). In one example, a secure mechanical lock may be achieved by restraining the efac within a constricting aperture of a size greater than the tensioned diameter but less than the untensioned diameter, such that the untensioned end of the elastomer acts as a restraint upon the aperture.

Force applying components can include marks indicating tension or stretch. The indicia may be formed from colorant, including any means for providing visual contrast, such as ink, dye, paint, or the like. Force applying components may also be disposable.

As noted above, it is generally desirable to use a non-reactive elastomeric force applying component such as a silicone, but silicone is normally difficult to secure. The viscoplastic properties of low durometer material, such as silicone, fall below the threshold where the material will hold a knot. Adequate constricting force may not be applied upon the material by the material itself to retain it under load because the application of the load reduces the material diameter beyond the minimum compression diameter of the constricting loop. This precludes the use of conventional surgical knot tying techniques because such knots will not hold. An additional complication is the tendency of the material to creep, or slip, when alternative capture methods are used. Thus, it is difficult to secure a silicone efac when a force is applied to the efac without the efac being cut or otherwise caused to fail by the securing structure.

Successful structures for securing a silicone elastomer (or other low durometer material) must clamp the silicone elastomer structure with enough force to hold it in place (avoiding creep) but with sufficiently distributed force that the elastomer is not severed. This invention provides structures that result in sufficient contact between an efac (including a silicone efac) and anchor structure that the two do not slide relative to each other while avoiding cutting or tearing the efac. Such structure can be provided by squeezing the efac between, or forcing it against, planar or relatively large radius arcuate surfaces while avoiding contact between the efac and arrises (intersections of planar surfaces) that might cut the elastomer.

Such a structure can be achieved with opposed planar or arcuate surfaces forming a Vee-shape and oriented so that tension on the efac forced into the gap between the surfaces will cause any reduction in outer diameter of the efac, such as occurs with added load, to result in the efac securing purchase lower in the Vee. In this manner, the efac-to-anchor structure contact is maintained, thereby improving the lock between the elastomer and anchor structure. Similarly, parallel surfaces may be engineered to provide an entrapment force and prescribed release tension for the efac in order to provide a maximum applicable tension and integral safety release.

The opposed surfaces can be provided by a variety of structures, such as arcuate surfaces provided by suitably rigid round wire or rod or by rounded opposed edges of plates of metal, plastic or other suitable material. Such structure can also be provided in other forms. For instance, the opposed surfaces between which the efac is trapped can also be provided by opposed flanges, typically positioned on a post or column and shaped so that the opposed flange surfaces get progressively closer together at points nearer the column. In such a structure, a first one of the opposed surfaces can be planar and can be, for instance, a flat base, provided that the other flange or other efac contact structure provides a surface that gets progressively closer to the first surface as the efac moves in the direction force applied to it during use will cause it to tend to move. For instance, the other flange can present a truncated conical surface.

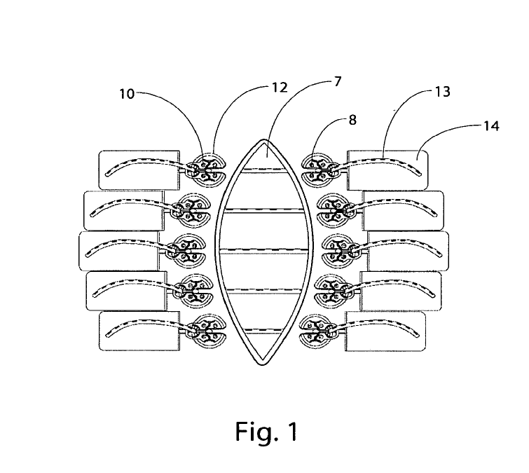

As shown in FIGS. 1-3, a button anchor 8 of this invention comprises an anchoring portion 10, which rests on an anchor pad 12 and which can optionally engage a load distributing anchor tail 14. This button anchor 8 remains external to the human or animal tissues, and comprises specific features for anchoring a fac traveling across a wound or through tissues that, by its presence and ability to apply a reducing force, provides the specific benefit of moving or moving and stretching tissue to bring reduction or closure of a full thickness wound where the wound margins lie beyond a distance where they can be primarily closed without undue force. In one example, a fac is passed through the skin, engaging or encircling the sub-dermal structures requiring closure, and returned through the skin on the other side of a wound or incision. The button anchors 8 are applied to the ends of the fac, allowing the fac to be tensioned and anchored, thereby applying a sub-dermal reduction force, as illustrated in FIG. 1. In an alternative embodiment, button anchors 8 positioned on opposite sides of a wound secure a fac that passes over the wound and that does not penetrate the tissue.

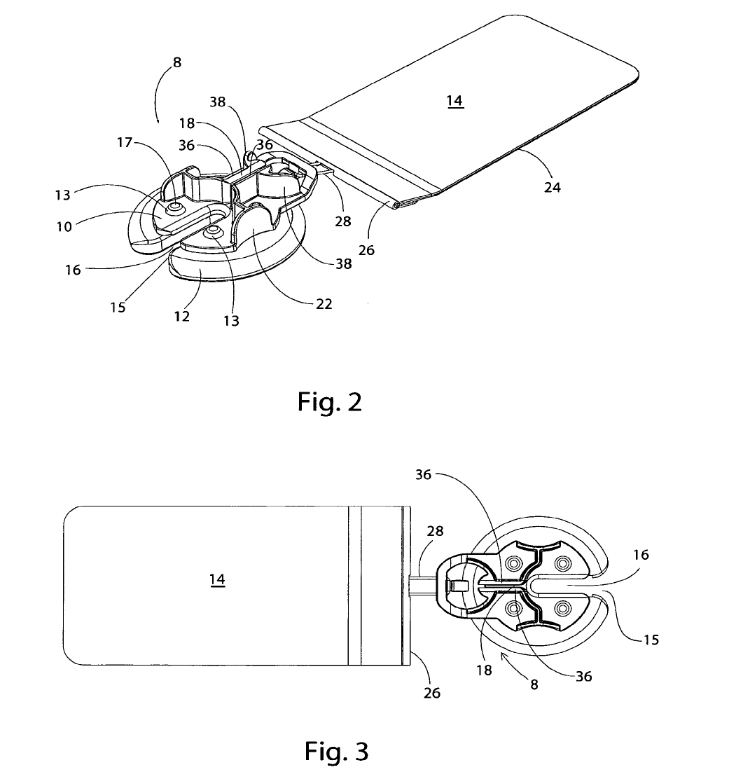

As shown in FIGS. 2-5, the anchoring portion 10 has a large slot 16 and a smaller slot 18 for engagement of an efac, such as an elastomer. Slot 18 includes walls 36 and is a metered tension, elastomer-locking slot, with a shape, length and size such that the slot 18 captures and anchors the elastomer but allows the elastomer to migrate if tension exceeds a pre-determined level, thereby creating a limit to the amount of force that can be applied by the system. This limit is determined at the time of manufacture of the anchoring portion 10 by controlling the relationship between the size of the slot 18 and the diameter or cross-sectional area of the elastomer. The cross-sectional area of the untensioned portion of the elastomer decreases as the elastomer elongates under increased tension. If a force applied to the elastomer exceeds the therapeutic force range, elongation and resulting reduction in diameter cause the elastomer to release within the slot, returning the quantity of tension to one within the therapeutic limit of the elastomer.

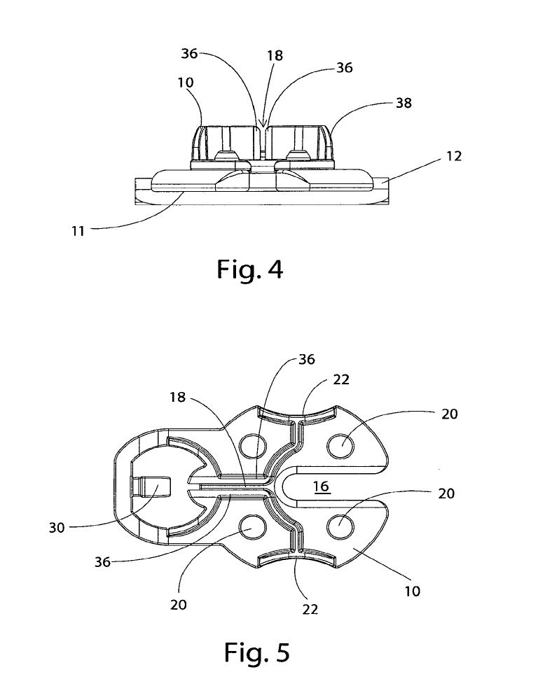

Convex upstanding regions 38 (visible in FIGS. 1 and 4) of the anchoring portion 10 prevent other objects from catching the edges of the button anchor 8.

The anchoring portion 10 may be molded of polycarbonate plastic or any other appropriately rigid and strong polymeric material suitable for use in the surgical applications for which the present invention is intended. Alternatively it may be molded, machined or otherwise formed or fabricated of any other suitably strong, surgically acceptable material such as stainless steel.

While the size of the button anchor 8 of this invention may be varied depending on the situation in which it is used, anchoring portion 10 may be approximately 32 mm in diameter. An anchoring portion 10 for use with an elastomeric three mm diameter, 40 durometer silicone cord may have a slot 18 one mm in width (i.e., the distance between walls 36), 7.3 mm in height and 11 mm in length. Many other dimensions are also usable provided that the desired coupling with elastomer is achieved (generally as described above).

Various arcuate or curved surface shapes for anchor efacs attachment structures are described above. It should be understood that functionally equivalent shapes can also be used, such as, for instance, a rod having a cross-section that is not curved but rather is a polygon.

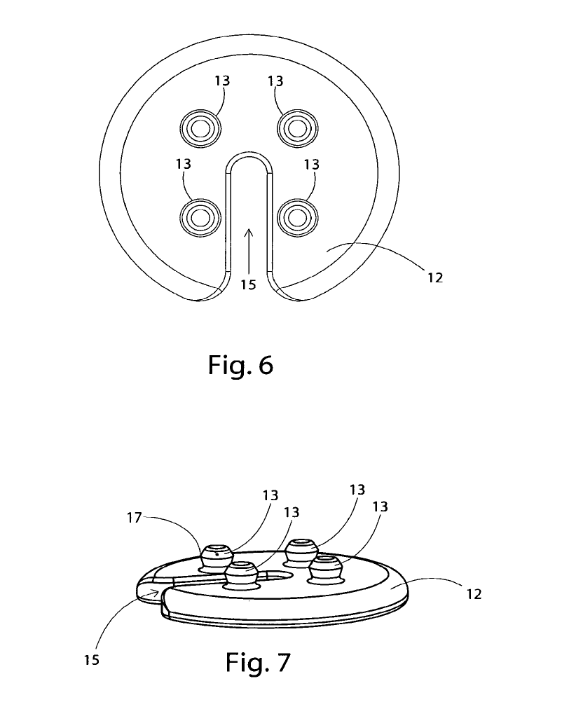

As shown in FIGS. 6 and 7, anchor pad 12 includes a slot 15 that corresponds to slot 16 of the anchoring portion 10. Anchor pad 12 dissipates the compression load exerted by one or more facs connected to the anchoring portion 10 over the surface of the patient's skin and works to prevent maceration or undue restriction of the underlying blood circulation. The anchor pad 12 is generally the same size and shape as the anchoring portion 10, but it may be smaller or larger in alternative embodiments. For example, larger pads may be used in patients with compromised skin tissues, including the elderly or those with associated co-morbidities, such as diabetes.

The anchor pad 12 may be made of a compressible material such as silicone, or any other suitable material. The skin contact surface (i.e., the underside) of anchor pad 12 may be smooth or it may be textured in order to accommodate fluid dissipation. The skin contact surface may be flat, convex, concave or multi-planar to accommodate anatomical contour. The skin-contacting surface of pad 12 may also be coated or treated to provide antimicrobial properties. In one embodiment, the skin-contacting surface of the anchor pad includes an adhesive.

As shown in FIG. 5, the anchoring portion 10 is penetrated by apertures 20 that secure the anchoring portion 10 to the anchor pad 12. Tabs 13 (shown in FIG. 7) project from anchor pad 12 and are received in apertures 20 of anchoring portion 10. Enlarged diameter end 17 of tabs 13 retain anchoring portion 10 on pad 12. In an alternative embodiment, the anchor pad 12 is adhered, adhesively bonded, or molded to anchoring portion 10. In one example, the anchor pad 12 and anchoring portion 10 are an integral unit.

As shown in FIGS. 2 and 5, finger grips 22 facilitate gripping and manipulating the button anchor 8 by opposed digits. Finger grips 22 are concave in the embodiment illustrated in the drawings, but the gripping portion may also be convex, multi-planar or textured.

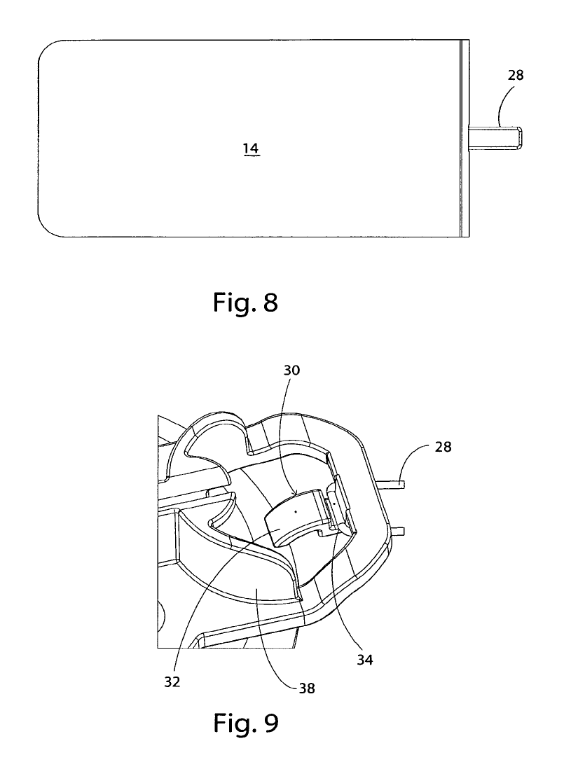

Optional anchor tail 14, shown in FIGS. 2, 3 and 8, may be utilized to further dissipate and distribute the shear-load placed on the skin by performing wound closure over the maximum possible surface area. In one embodiment, the anchor tail 14 is formed from polyurethane foam having an adhesive for attachment to the skin and includes a wire that forms a loop 28 at end 26. In alternative embodiments, the anchor tail 14 may be formed from any suitable fabric, foam or film. Such material may be elastic or inelastic. Preferably the anchor tail 14 material conforms to the skin surface and mimics the elasticity of the skin. In addition, the loop 28 may be formed or molded as a separate or integral component.

Anchoring portion 10 of button anchor 8 includes structure for engaging anchor tail 14. Such structure may include a hole, tab, cleat or other suitable structure. In one embodiment, shown in the Figures, and particularly in FIG. 9, the anchoring portion 10 includes a hook 30 having a ramp 32 for guiding the wire loop 28 of tail 14 up and into depression 34 of anchoring portion 10. In use, the anchor tail 14 is attached to the anchoring portion 10 via the engagement hook 30 and is adhered to the skin. In this manner, anchor tail 14 bolsters the button anchor 8 and dissipates the forward force load (a force vector that travels toward the wound edge and parallel to the skin surface) over a large area of healthy skin located behind the button anchor 8. While the hook 30 and loop 28 provide one example of structure to couple the anchor tail and anchor, any suitable structure may be used.

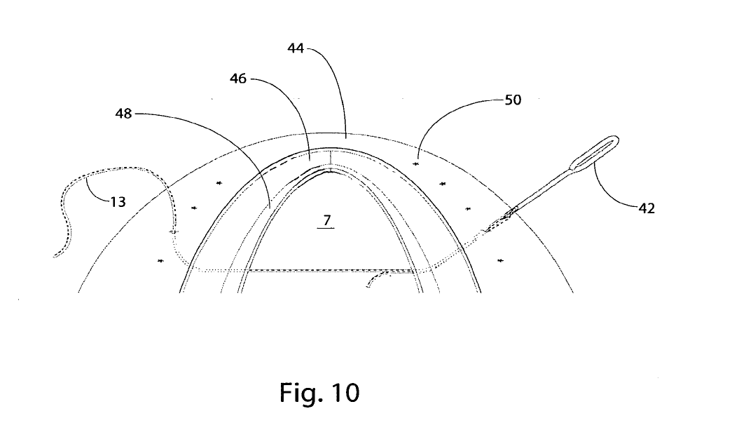

The system of this invention may be used to provide deep fascia repair and deep fascia dynamic wound reduction. In one embodiment, illustrated in FIG. 10, a silicone elastomer 13 is coupled to a cannula-like device 42 and is passed through the dermis 44, fat layer 46, and fascia 48 at an optional anchor placement mark 50 placed on the skin prior to installation of the system. After passing through the area of the wound 7, the elastomer 13 is presented through slot 16 of anchoring portion 10 and slot 15 of anchor pad 12 of button anchor 8, where it is then captured and secured in smaller slot 18 of anchoring portion 10. In this manner, closure force is applied to a wound or incision 7. Multiple sets of anchors and elastomers may be used, as shown in FIG. 1.

The elastomer 13 may either be presented through the skin and through the slot 16 of an anchor previously placed, or the elastomer 13 may exit the skin, at which time the slot 16 and the pad slot 15 of the anchor 8 may be moved into place around the elastomer 13. The efac may be used to apply tension to sub-dermal structures (deep fascia) but the efac tension may be adjusted from above the skin by increasing or decreasing the tension at the smaller slot 18. The anchor 8 acts as a grommet, removes the point load from the exit hole to reduce the occurrence of localized failures, and also allows adjustment of the tension across the wound. In this manner, the anchor bolsters the perimeter of the transcutaneous opening through which the elastomer passes, reducing localized failures and also reducing scarring.

A system according to this invention may provide wound stabilization of abdominal procedures. For example, this system may be used to restore radial abdominal integrity during prolonged interventions for complications such as abdominal infections management or which require large abdominal access. This system increases patient comfort and mobility by providing abdominal containment and support, and maintains normal skin tensions during intervention to minimize retraction.

Another system of this invention may provide stability to sternal or chest non unions as can arise after open heart surgical procedures. In addition, systems of this invention may be used with conventional primary wound closure methods to distribute skin system tensions to healthy skin beyond the wound, thereby minimizing stress at the wound site and reducing dehiscence. A system of this invention may be applied pre-operatively to tension skin and create surplus tissue, allowing excisions to be covered and closed in a conventional manner. Embodiments of this invention may also be used as a dressing retention system by providing efac lacing across the wound site, which passes over the wound dressing and secures it in position. Adhesives may be used on the skin contacting surface of the anchor pad but such adhesives normally would not be required, thereby further facilitating the periodic inspection and cleaning of tissues under the anchor pads.

All of the tissue attachment structure and anchor designs described herein may be produced in a variety of sizes.

The systems and methods of moving or moving and stretching plastic tissue according to this invention are not confined to the embodiments described herein but include variations and modifications within the scope and spirit of the foregoing description and the accompanying drawings. For instance, the scale of the components of the invention can vary quite substantially depending on the nature and location of the tissue with which the invention is used. The configuration of the tissue attachment structures can also be varied for the same reasons and for aesthetic reasons. While most of the elements of the illustrative embodiments of the anchors of this invention depicted in the drawings are functional, aspects of the shape and appearance of the illustrative embodiments are nonfunctional and ornamental.

The materials from which the components used in practicing this invention are made can be those described above as well as others, including materials not yet developed that have appropriate properties of strength, elasticity and the like that will be apparent to those skilled in the art in light of the foregoing. For instance, useful materials generally must be sterile or sterilizable and non-reactive. The illustrated components are typically intended to be disposable, but the invention can also be practiced using reusable components.

1. A system for moving tissue comprising:
(a) at least one non-reactive force applying component; and
(b) at least one anchor for attachment to the tissue, the anchor comprising
(i) a first slot sized to allow the force applying component to freely pass through the anchor, and
(ii) a second slot sized to capture the force applying component without knotting or tearing the force applying component and formed to provide adjustable attachment of the force applying component; and
(c) at least one anchor pad coupled to the at least one anchor and adapted to distribute force across an area of the tissue, the pad comprising a pad slot corresponding to the first slot of the anchor.

2. The system of claim 1, further comprising an anchor tail.

3. The system of claim 2, wherein the anchor tail further comprises adhesive and an engaging loop.

4. The system of claim 2 wherein the anchor tail further comprises polyurethane foam.

5. The system of claim 2 wherein the anchor tail further comprises fabric.

6. The system of claim 5 wherein the fabric is elastic and conforms to the skin surface.

7. The system of claim 3 wherein the loop comprises wire.

8. The system of claim 3, wherein the anchor further comprises a hook having a ramp for guiding the loop of the anchor tail up and into a depression of the anchor.

9. The system of claim 1, wherein the second slot of the anchor is a metered tension, locking slot, with a shape, length and size such that the slot captures the force applying component but allows the component to migrate if tension exceeds a pre-determined level.

10. The system of claim 1 wherein the force applying component comprises elastomer.

11. The system of claim 10 wherein the elastomer comprises silicone.

12. The system of claim 1 wherein the tension is adjustable within an elastic limit of the force applying component.

13. The system of claim 1 wherein the force applying component is adapted to deform to be released from the anchor upon application of a predetermined force.

14. The system of claim 1 wherein at least two anchors are adapted to be attached to the tissue on opposite sides of a wound or incision.

15. The system of claim 14 wherein the at least two anchors secure at least one force applying component and wherein the force applying component passes through tissue and fascia.

16. The system of claim 1 wherein the anchor pad further comprises a compressible material.

17. The system of claim 1 wherein the anchor pad further comprises silicone.

18. The system of claim 1, the anchor pad further comprising a skin contacting surface having antimicrobial properties.

19. The system of claim 1 wherein the anchor further comprises apertures and the anchor pad further comprises projections that pass through the apertures and couple the anchor pad to the anchor.

20. The system of claim 1 wherein the anchor and anchor pad are adhesively bonded.

21. The system of claim 1 wherein the anchor and anchor pad are integral.

22. The system of claim 1, wherein the anchor further comprises finger grips.

23. The system of claim 1 wherein the tissue to be moved is healthy tissue.

24. A system for moving tissue comprising:
(a) at least one non-reactive force applying component; and
(b) at least one anchor for attachment to the tissue, the anchor comprising an opening sized to allow the force applying component to freely pass through the anchor, wherein the anchor distributes force applied to the tissue and bolsters a perimeter of a transcutaneous opening through with the force applying component passes.

25. The system of claim 24 wherein the anchor further comprises
(i) a first slot sized to allow the force applying component to freely pass through the anchor, and
(ii) a second slot sized to capture the force applying component without knotting or tearing the force applying component and formed to provide adjustable attachment of the force applying component.

26. The system of claim 24 further comprising at least one anchor pad.

27. The system of claim 26 wherein the at least one anchor pad is coupled to the at least one anchor and comprises a pad slot corresponding to the first slot of the anchor.

28. The system of claim 24, further comprising at least one anchor tail.

29. The system of claim 28 wherein the at least one anchor tail comprises adhesive for attachment to the tissue and wherein the anchor tail is coupled to the anchor.

30. An anchor assembly for attachment to tissue to transmit force for moving the tissue, the anchor assembly comprising:
(a) at least one anchor for attachment to the tissue, the anchor comprising
(i) a first slot sized to allow a force applying component to freely pass through the anchor,
(ii) a second slot sized to capture the force applying component without knotting or tearing the force applying component and formed to provide adjustable attachment of the force applying component, and
(b) an anchor tail attachable to the anchor and comprising adhesive for attachment to the surface of the skin.

31. The anchor assembly of claim 30, wherein the anchor tail further comprises a loop.

32. The anchor assembly of claim 30, further comprising an anchor pad.

33. The anchor assembly of claim 32, further comprising a slot sized to allow a force applying component to freely pass through the anchor.

34. An anchor assembly for attachment to tissue to transmit force for moving the tissue, the anchor assembly comprising:
(a) at least one anchor for attachment to the tissue, the anchor comprising
(i) a first slot sized to allow a force applying component to freely pass through the anchor,
(ii) a second slot sized to capture the force applying component without knotting or tearing the force applying component and formed to provide adjustable attachment of the force applying component, and
(iii) a hook for coupling the anchor tail to the anchor; and
(b) an anchor pad adapted to distribute force across an area of the tissue comprising a pad slot corresponding to the first slot of the anchor; and
(c) an anchor tail comprising adhesive for attachment to the surface of the skin and a loop for engaging the anchor.

35. The anchor of claim 34 wherein the anchor tail further comprises polyurethane foam.

36. The anchor of claim 34 wherein the anchor tail further comprises elastic fabric.

37. The anchor of claim 34 wherein the loop of the anchor tail comprises wire.

38. The anchor of claim 34, the anchor further comprising a depressions and the anchor hook further comprising a ramp for guiding the loop of the anchor tail up and into the depression of the anchor.

39. The anchor of claim 34, wherein the second slot of the anchor is a metered tension, locking slot, with a shape, length and size such that the slot captures a force applying component but allows the force applying component to migrate if tension exceeds a pre-determined level.

40. The anchor of claim 34, wherein the anchor pad further comprises silicone.

41. The anchor of claim 34, the anchor pad further comprising a skin contacting surface having antimicrobial properties.

42. The system of claim 34 wherein the anchor further comprises apertures and the anchor pad further comprises projections that pass through the apertures and couple the anchor pad to the anchor.

43. The system of claim 34 wherein the anchor and anchor pad are adhesively bonded.

44. The system of claim 34 wherein the anchor and anchor pad are integral.

45. The system of claim 34, wherein the anchor further comprises finger grips.

46. A method for moving and stretching plastic tissue comprising:
(a) threading a force applying component through the skin and through muscle or fascia, the force applying component exiting the skin on the opposite side of a wound or incision,
(b) securing a first end of the force applying component to a first anchor and securing a second end of the force applying component to a second anchor without knotting or tearing the force applying component;
(c) adjusting tension by removing and re-securing the at least one force applying component to the at least one anchor.

47. The method of claim 32 wherein the adjusting tension further comprises referring to a quantitative tension indication feature of the force applying component.
